(19) United States
(12) Patent Application Publication
(10) Pub. No.: US 2015/0246100 A1
(43) Pub. Date: Sep. 3, 2015

(54) Modulation of Immune Function by Dietary Bovine Lactoferrin

(71) Applicants: THE BOARD OF TRUSTEES OF THE UNIVERSITY OF ILLINOIS, Urbana, IL (US); Nestec S.A., Vevey (CH)

(72) Inventors: Sharon M. Donovan, Champaign, IL (US); Nikhat Contractor, Bryn Mawr, PA (US); Sarah S. Comstock, East Lansing, MI (US)

(21) Appl. No.: 14/433,207

(22) PCT Filed: Oct. 3, 2013
(86) PCT No.: PCT/US2013/063242

§ 371 Date: Apr. 2, 2015

Related U.S. Application Data
(60) Provisional application No. 61/709,242, filed on Oct. 3, 2012.

(51) Int. Cl. A61K 38/40

(52) U.S. Cl. CPC A61K 38/40

(57) ABSTRACT

The invention provides methods of increasing immune cell function in a newborn mammal that has not consumed any substantial amounts of colostrum or breast milk comprising administering an infant formula comprising about 1.0 to about 10 g/L of lactoferrin to the newborn mammal.

### PRIORITY

This application claims the benefit of U.S. Ser. No. 61/709,242, which was filed on Oct. 3, 2012, which is incorporated by reference in its entirety herein.

### BACKGROUND OF THE INVENTION

Formula-fed infants have higher incidences of infectious diseases and other immune-related diseases including allergy and autoimmune diseases than breastfed infants. In the first year of life, after adjusting for confounders, there were 2033 excess office visits, 212 excess days of hospitalization, and 609 excess prescriptions due to lower respiratory tract illnesses, otitis media, and gastrointestinal illness per 1000 never-breastfed infants compared with 1000 infants exclusively breastfed for at least three months. These additional health care services cost the managed care health system between $331 and $475 per never-breastfed infant during the first year of life. Therefore, there is a need to reduce infectious diseases and improve immune development of formula-fed infants.

### SUMMARY OF THE INVENTION

One embodiment of the invention provides a method of increasing immune cell function in a newborn mammal that has not consumed any colostrum or breast milk. The method comprises administering an infant formula comprising about 1.0 to about 10 g/L of bovine lactoferrin to the newborn mammal. The infant formula can comprise about 1.0 to about 3.6 g/L of or about 3.6 to about 5.0 g/L of bovine lactoferrin. The newborn mammal can have immature immune function, can be permanently immunocompromised, can be temporarily immunocompromised, can be fully gestated or can be born prematurely. The newborn mammal can be just born to 1 hour old, can be just born to about 5 hours old or can be just born to about 1 year old. The newborn mammal can have a primary or secondary immunodeficiency such as a B-cell defect, a T-cell disorder, a combined B-cell and T-cell defect, a natural killer cell defect, a phagocytic cell defect, a complement system deficiency, malnutrition, can be on immunosuppressive medications, can have cancer, a chronic infection, diabetes, a hepatic insufficiency, hepatitis, lymphangiectasia, aplastic anemia, graft v. host disease, sickle cell disease, radiation therapy, splenectomy, cytomegalovirus, Epstein-Barr virus, measles virus, varicella-zoster virus, nephrotic syndrome, renal insufficiency, uremia, or AIDS. The newborn mammal can be a human having an IgG concentration of less than about 7 g/L. The mother of the newborn infant can have placental abnormalities, hypergammaglobulinemia, HIV infection, placental malaria, a humoral immunedeficiency, or other infection or disease that causes reduced placental transfer of IgG. The increase in immune cell function can be an increase in total serum immunoglobulin concentration or an increase in cytokine secretion from immune cells or a combination thereof. The increase in cytokine secretion can be an increase in IL-6, IL-10, IFN-gamma, IL-4, TNF-alpha, IL12p40, or a combination thereof. The newborn mammal can be a human, pig, canine, equine, or feline.

Another embodiment of the invention provides a method of increasing immune cell function in a newborn mammal that has consumed colostrum or breast milk for a period not exceeding 1 week after birth, comprising administering an infant formula comprising about 1.0 to about 10 g/L of bovine lactoferrin to the newborn mammal.

Yet another embodiment of the invention provides a method of increasing immune cell function in a newborn mammal that has consumed a total not exceeding about 2 ml of colostrum, or a total not exceeding about 10 ml of breast milk, or a total not exceeding about 2 ml of colostrum and about a total not exceeding about 10 ml of breast milk, comprising administering an infant formula comprising about 1.0 to about 10 g/L of bovine lactoferrin to the newborn mammal.

### BRIEF DESCRIPTION OF THE DRAWINGS

FIG. 1 shows the effect of 1.0 g/L lactoferrin (LF1) or 3.6 g/L lactoferrin (LF3) on serum IgG levels.

FIGS. 2A-C show the effect of dietary lactoferrin on the production of IL-6 by stimulated and unstimulated mesenteric lymph node cells.

FIGS. 3A-B show the effect of dietary lactoferrin on the production of IL-10 by stimulated and unstimulated mesenteric lymph node cells.

FIGS. 4A-B show the effect of dietary lactoferrin on the production of IFN-gamma by stimulated and unstimulated mesenteric lymph node cells.

FIG. 5 shows the effect of dietary lactoferrin on the production of IL-4 by stimulated and unstimulated mesenteric lymph node cells.

FIGS. 6A-B show the effect of dietary lactoferrin on the production of IL-10 by stimulated and unstimulated spleen cells.

FIGS. 7A-B show the effect of dietary lactoferrin on the production of IFN-gamma by stimulated and unstimulated spleen cells.

FIG. 8 shows the effect of dietary lactoferrin on the production of IL-4 by stimulated spleen cells.

FIG. 9 shows the effect of dietary lactoferrin on the production of TNF-alpha by stimulated spleen cells.

FIG. 10 shows the effect of dietary lactoferrin on the production of IL12p40 by stimulated and unstimulated spleen cells.

### DETAILED DESCRIPTION OF THE INVENTION

As used herein, the singular forms “a,” “an,” and “the” include plural referents unless the context clearly dictates otherwise. The term “about,” when used in conjunction with a numerical value means the numerical value can vary plus or minus by 5% of the numerical value.

The invention provides methods for increasing immune cell function in infants and in particular infants that have received substantially no colostrum or breast milk, are preterm, immunocompromised, unhealthy, have immature immune function, or combinations thereof. The method comprises providing dietary lactoferrin in an infant formula to the infant.

Infants can be any mammal including, e.g., human, pig, canine, equine, or feline. Infants can be just born, 1, 5, 10, 24, 36, 48, 60, 72, or more hours old, 1, 2, 3, 4, 5, 10, 15, 20, 30, 40, 50 weeks or more old, or about 1 year old for any value between just born and about 1 year old). The infant can be just born to about 1, 5, 10, 24, 36, 48, 60, 72, or more hours old, 1, 2, 3, 4, 5, 10, 15, 20, 30, 40, 50 weeks or more old, or about 1 year old (or any range between just born and about 1 year old).

In one embodiment an infant receives no colostrum or breast milk from its mother or any other source at any time. Optionally, the infant has received no colostrum after 1, 2, 3, 4, 5, 10, or 12 hours after birth. Optionally, the infant has received no breast milk after 1, 2, 3, 4, 5, 6, days or 1, 2, 3, or 4 weeks after birth. In another embodiment, the infant has consumed, in total (over the entire life of the infant), less than about 1, 2, 3, 5, 7, 10, 15, 20, 30, or 50 ml of colostrum, or less than about 5, 10, 15, 20, 30, 40, 50, 60, 70, 80, 90, 100, 150, 200, 250, or 300 ml of breast milk, or less than about 1, 2, 3, 5, 7, 10, 15, 20, 30, or 50 ml of colostrum and less than about 5, 10, 15, 20, 30, 40, 50, 60, 70, 80, 90, 100, 150, 200, 250, or 300 ml of breast milk. Optionally, the infant has received no breast milk for a period not exceeding about 1, 2, 3, 4, 5, 6, days or about 1, 2, 3, or 4 weeks after birth. In another embodiment, the infant has consumed a total (over the entire life of the infant) not exceeding about 1, 2, 3, 5, 7, 10, 15, 20, 30, or 50 ml of colostrum, or a total not exceeding about 5, 10, 15, 20, 30, 40, 50, 60, 70, 80, 90, 100, 150, 200, 250, or 300 ml of breast milk, or a total not exceeding about 1, 2, 3, 5, 7, 10, 15, 20, 30, or 50 ml of colostrum and a total not exceeding about 5, 10, 15, 20, 30, 40, 50, 60, 70, 80, 90, 100, 150, 200, 250, or 300 ml of breast milk. Colostrum is a form of milk produced by the mammary glands of mammals (including humans) in late pregnancy. Most mammals will generate colostrum just prior to giving birth. Colostrum contains antibodies to protect the newborn against disease, as well as being lower in fat-and higher in protein than ordinary breast milk. Colostrum is produced by a mother for about 24 to about 72 hours after birth. Breast milk is the milk produced by the breasts or mammary glands of a female mammal for her infant offspring. Breast milk is produced about 24 to 72 hours after giving birth, that is, after the period of producing colostrum has passed.

Infant formula is a nutritional composition for mammalian infants and contains sufficient calories, protein, carbohydrate, fat, vitamins, and minerals to serve as a sole source of nutrition when provided in sufficient quantity to the infant.

The infant formula can be a cow's milk formula, a soy protein based formula, a partially hydrolyzed formula, an extensively hydrolyzed formula, a hypoallergenic infant formula made from individual amino acids, a lactose-free formula, an anti-regurgitation formula (which thickens in the stomach), or an acidified cow's milk formulation. Infant formulas can comprise: protein, fat, linoleic acid, vitamin A, vitamin C, vitamin D, vitamin E, vitamin K, thiamin, riboflavin, vitamin B₆, vitamin B₁₂, niacin, folic acid, pantothenic acid, calcium, magnesium, iron, zinc, manganese, copper, phosphorus, iodine, sodium chloride, potassium chloride, carbohydrates (e.g., lactose, sucrose, glucose, natural starches, modified starches), nucleotides, stabilizers, emulsifiers, biotin, choline, inositol, diluent (e.g., skim milk or water).

Infant formulas can be prepared in any product form suitable for use in infants, including for example, reconstitutable powders, ready-to-feed liquids (liquid forms suitable for administration to an infant, including reconstituted powders, diluted concentrates, and manufactured liquids and dilatable liquid concentrates). Infant formula product forms are well known in the art.

Infant formula can be in a liquid form or a powder form for reconstitution. Powder infant formulas can be in the form of flowable or substantially flowable particulate compositions that can be easily scooped and measured with a scoop or similar other device. The powder compositions can easily be reconstituted with a suitable aqueous fluid, e.g., water, to form a liquid nutritional formula for immediate oral or enteral use (e.g., use in right after reconstitution, within about 24 hours or within about 48 hours). Powder formulations include spray dried, agglomerated, dry mixed or other known or otherwise effective particulate forms. The quantity of a nutritional powder required to produce a volume suitable for one serving can vary.

The infant formulas for use herein (powder and liquid forms) can be packaged and sealed in single or multi-use containers, and then stored under suitable conditions for about 12, 24, 36 or more months. Multi-use containers can be opened and than covered for repeated use provided that the covered package is stored under suitable conditions and the contents used within about 1, 2, or 3 days (liquid forms) or one month (powder forms).

An infant can be fed about 50, 75, 100, 200, 300, 400, 500, ml/kg of body weight/day of formula with or without the addition of solid food. An infant can be fed formula about 1, 2, 3, 4, 5, 10 or more times a day.

Lactoferrin binds to receptors on the surface of mammalian cells: cells of the innate (NK cells, neutrophils, macrophages, basophils, neutrophils, and mast cells) and adaptive (lymphocytes, and antigen-presenting cells) immune systems, and also epithelial and endothelial cells. Through these interactions, lactoferrin is able to modulate the maturation and functions of immune cells and, thus, to influence both adaptive and innate immunities. Infant formula of the invention can comprise about 0.25, 0.5, 0.75, 1.0, 2.0, 3.0, 3.5, 3.6, 4.0, 5.0, 6.0, 7.0, 8.0, 9.0, 10.0, 11.0, 12.0, 13.0, 14.0, 15, or more g/L of lactoferrin (or any range or value between about 0.25 and about 15 g/L). In one embodiment of the invention the infant formula comprises about 1.0 to about 10 g/L, about 1.0 to about 3.6 g/L, about 3.6 to about 5.0 g/L, or about 3.6 to about 10 g/L of lactoferrin. The lactoferrin can be bovine lactoferrin.

In one embodiment of the invention, the infant is permanently or temporarily immunocompromised (e.g., immunocompromised for about 1 day, 1, 2, 3, weeks, 1, 2, 3, 4, months or more), or has immature immune function. The infant can be immunocompromised due to primary immune deficiency (a deficient, absent, or defective immune system due to genetic defect) e.g., B-cell defects, T-cell disorders, combined B-cell and T-cell defects, natural killer cell defects, phagocytic cell defects, or complement system deficiencies. The immunodeficiency can be a secondary immunodeficiency due to, e.g., malnutrition, use of immunosuppressive medications, cancer (e.g. leukemia, lymphoma, multiple myeloma), chronic infections, diabetes, hepatic insufficiency, hepatitis, lymphangiectasia, aplastic anemia, graft v. host disease, sickle cell disease, radiation therapy, splenectomy, cytomegalovirus, Epstein-Barr virus, measles virus, varicella-zoster virus, nephrotic syndrome, renal insufficiency, uremia, or AIDS.

In one embodiment, an infant has physiologic immunodeficiency of a normal, full-term, healthy infant or a more pronounced physiologic immunodeficiency due to preterm birth, an immunocompromised state, illness or other issue. A normal, full-term, healthy infant is born with physiologic immunodeficiency because their innate and adaptive immunological responses are greatly suppressed. The physiologic immunodeficiency is more pronounced in preterm, immunocompromised, and unhealthy infants. Physiologic immunodeficiency includes low levels of IgG and immature T cells (resulting in, e.g., lower levels of production of cytokines), immature cytotoxic activity, immature neutrophil production and function, immature complement levels, or combinations thereof. An infant with a more pronounced physiological immunodeficiency has 10, 20, 30, 40, 50% or lower levels of IgG IgM, and/or cytokines, 10, 20, 30, 40, 50% or more immature T cells, immature cytotoxic activity, immature neutrophil production and function, immature complement levels, or combinations thereof as compared to a normal, full term, healthy infant.

Maternal IgG is normally transferred to the fetus via the placenta during the third trimester of pregnancy. The IgG lasts for about 4-6 months after the infant is born. Where an infant is born prematurely the infant will have low levels of IgG because the full amount of IgG was not received from the mother. Normal levels of IgG for a full-term infant are about 8-12 g/L. Low levels of IgG are about 7.5, 7, 6, 5, 4, 3, 2 g/L, or less of IgG for a human.

In one embodiment of the invention, the infant is born to a mother with placental abnormalities, hypergammaglobulinemia, HIV infection, placental malaria, a humoral immunedeficiency, or other infection or disease that causes reduced placental transfer of IgG.

A premature human infant is born at less than about 37 weeks gestation, typically from about 26 to about 34 weeks gestation. Other mammals are born prematurely when they are born at less than about 93% of full gestation time.

A low birth weight human infant (term or premature) weighs less than about 2.5, 2.25, 2.0, 1.8, 1.5 kg or less at birth. Other mammals are low birth weight when they are born at less than about 75% of average weight of full term infants for that species.

Maternal antibodies provide passive immune protection to newborns and also actively influence the immune systems of newborns. Maternal antibodies can shape the B-cell repertoire of offspring and modulate the adult antibody responses of their offspring. For example, newborns of certain idiotype-immunized mothers or anti-idiotype treated newborns have suppressed expression for the certain idiotype for variable time periods. See, Rueff-Joy et al., J. Immunol. 16:721 (1998). Additionally, mothers immunized with a certain antigen produce newborns with large amounts of IgM antibodies with the same antigenic specificity and the same idiotype as the mother's antibodies. See id. This immune imprint can persist to the F₂ generation. Lemke et al., Eur. J. Immunol. 24:3025 (1994). IgA from mother's milk delays the development of germinal centers in GALT and the concentration of serum IgA. Furthermore, serum immunoglobulins stimulate B-cell development in offspring without modifying the B-cell repertoire. Additionally, immunoglobulins in mother's milk or colostrum can induce and maintain tolerance of Ck-specific CD8+ cytotoxic T lymphocytes. See, Rueff-Joy et al., J. Immunol. 16:721 (1998).

Infants who receive substantially no colostrum or breast milk or substantially no colostrum and substantially no breast milk and who optionally are pre-term and/or unhealthy (e.g., immunocompromised) do not receive the benefit of this immune system priming. It is unexpected that infants that received substantially no colostrum and/or breast milk and who optionally are pre-term and/or unhealthy would respond favorably to lactoferrin in infant formula because their immune systems have not been primed by colostrum or breast milk. Substantially no colostrum is less than 5 ml of colostrum. Substantially no breast milk is less than 10 ml of breast milk.

One embodiment of the invention provides a method of increasing immune cell function in a newborn mammal that has consumed substantially no colostrum, substantially no breast milk, or substantially no colostrum and substantially no breast milk comprising administering an infant formula comprising about 1.0 to about 10 g/L of bovine lactoferrin to the newborn mammal. Immune cells can be, e.g., T-cells, B-cells, or natural killer cells, or any other cells with antigen-presentation capability. Cells having antigen-presentation capability are cells that present protein or protein fragment complexes on their surface such that T or B cells that come into contact with such a complex become activated. A newborn mammal can have immature immune function, can be permanently immunocompromised, or can be temporarily immunocompromised. The newborn mammal can have been born prematurely or can be unhealthy. The increase in immune cell function can be an increase in total serum immunoglobulin concentration (e.g., an increase in total serum immunoglobulin concentration or an increase in cytokine secretion from immune cells or a combination thereof). The increase in cytokine secretion can be an increase in IL-6, IL-10, IFN-gamma, IL-4, TNF-alpha, IL12p40, or a combination thereof. The increase in total serum immunoglobulin concentration or increase in cytokine secretion can be an increase of about 5, 10, 20, 25, 30, 40, 50, 60, 70, 80, 90, 100, 200, 300, 400, 500, 600, 700, 800, 900, 1,000 percent or more (or any range or value between about 5 and about 1,000 percent) as compared to a similar infant who does not receive formula supplemented with lactoferrin.

All patents, patent applications, and other scientific or technical writings referred to anywhere herein are incorporated by reference herein in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### EXAMPLES

### Example 1

Newborn, colostrum-deprived piglets were fed formula containing 0.4 (control), 1.0 (LF1) or 3.6 (LF3) g/L bovine lactoferrin for 7 days or 14 days. Serum and immune tissues (spleen, ileal Peyer's Patches and mesenteric lymph nodes) were collected. Immune cells were isolated from blood, ileal Peyer's Patches, and mesenteric lymph nodes and unstimulated or stimulated ex vivo for 72 hours with polymyxin B (PB) (10 μg/ml), LF (50 μg/ml) +PB, LPS (2 μg/ml), LPS+LF, PreLPS+LF (combined 1 hour prior to addition to culture), or PHA (2.5 μg/ml).

Serum immunoglobulin concentrations were measured using porcine-specific antibody quantification sets from Bethyl Laboratories (Montgomery, Tex.). Briefly, 96 well, flat bottomed ELISA plates were coated with 10 μg/ml anti-pig capture antibody diluted in coating buffer (Carb/Bicarb, pH 9.6) and incubated overnight at 4 degrees C. The next day the coating buffer was removed and plates were blocked with 300 μl of 3% BSA/PBS at room temperature for 1 hour. After 1 hour, plates were washed 3 times with PBS/0.1% Tween™ 20 (polysorbate). Then, the samples were diluted in PBS/0.5% fish gelatin and 100 μl of each diluted sample was added to the wells. After one hour of incubation, the plate was again washed with PBS/Tween™ (polysorbate). Next, 100 μL of goat anti-pig IgG conjugated to horseradish peroxidase diluted to 1:75,000 in PBS/0.5% fish gelatin was added to each well. The plate was incubated in the dark for 1 hour. Next the plate was washed 5 times with PBS/Tween™ (polysorbate) and TMB (100 μl) was pipetted into each well. The plate was incubated in the dark for 15 minutes. Finally, 100 μL of 2N sulfuric acid was added as the stop solution. The plate absorbance was read at 450 nm on a SpectraMax with 570 nm as the plate correction wavelength. Data was calculated using the standard curve included in the quantification set.

To measure cytokine production, cells were isolated from the spleens and mesenteric lymph nodes (MLN) of 7-day-old piglets using a combination of enzymatic and mechanical dissociation methods. Total cell isolates were left un-stimulated or stimulated incubated for 72 hr at 36 degrees C. with 5% carbon dioxide. Stimulants included: polymixin B (PB, 10 μg/ml), LF (50 μg/ml)+PB, LPS (2 μg/ml), LPS+LF, PHA (2.5 μg/ml) or LF that had been incubated with LPS for 1 hour prior to addition to cultures. LPS stimulates B cells and other antigen presenting cells that express TLR4 (the LPS receptor). LPS does not stimulate T cells. PHA stimulates T cells. PHA binds to glycoproteins on the surface of T cells causing T cell activation.

Supernatants were collected at 72 hr, frozen and shipped to Panomics (Affymetrix, Santa Clara, Calif.) for analysis using the Procarta Cytokine Plex Kits on the Luminex Platform.

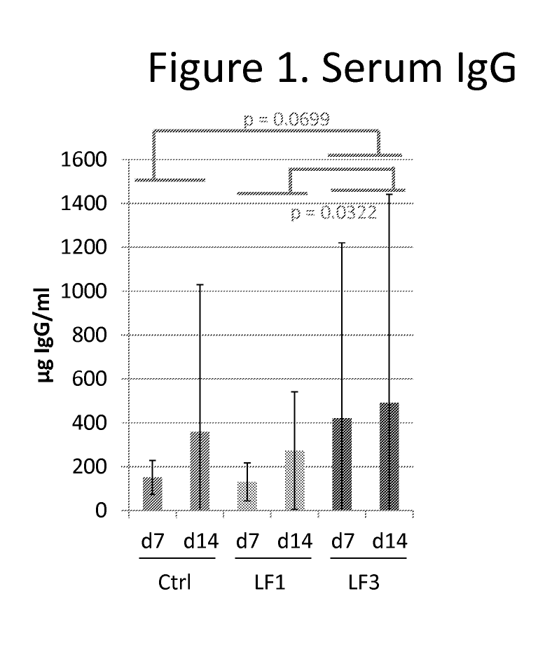

FIG. 1 demonstrates that serum IgG was increased with dietary lactoferrin. The LF3 treatment resulted in significantly increased serum IgG as compared to the LF1 treatment.

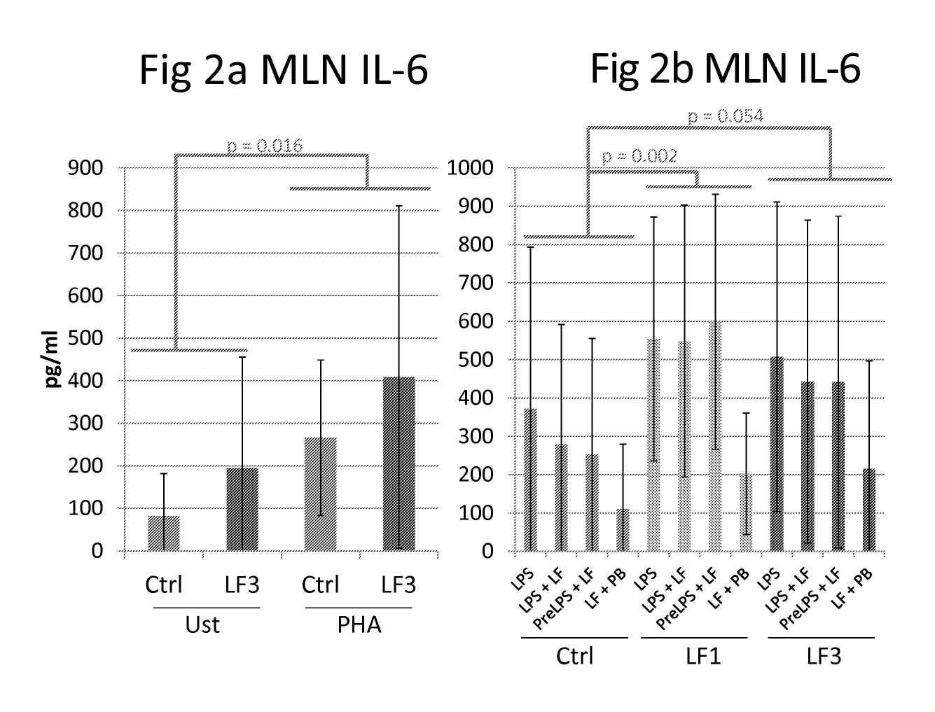

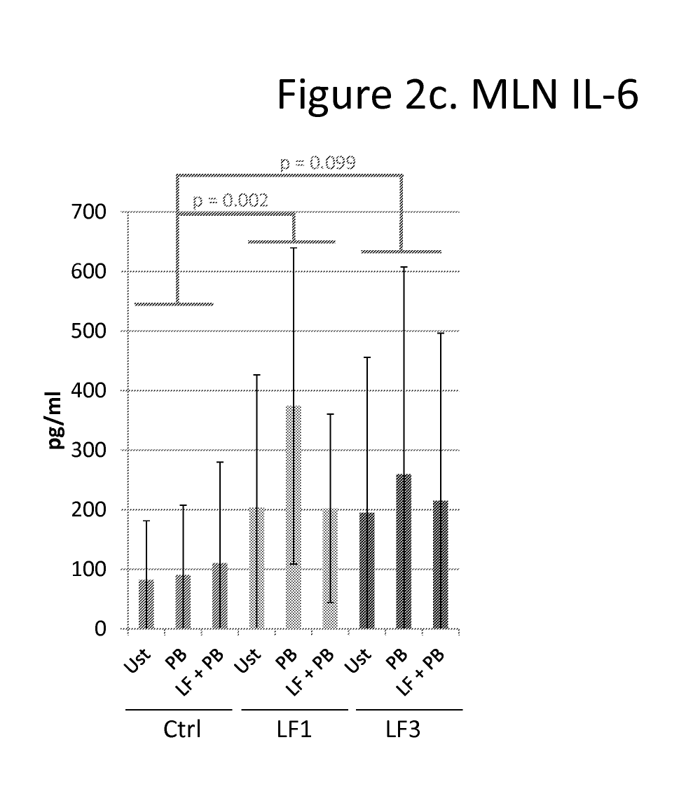

In mesenteric lymph node cells, dietary lactoferrin increased IL-6 production by ex vivo LPS-stimulated cells, but not PHA-stimulated cells. FIG. 2A. This indicates that lactoferrin must be orally administrated to be effective at increasing cytokine production and may not be effective if administered via another route such as intravenous or subcutaneously or in any other manner. Additionally, the differential response to the B cell stimulator LPS and the T cell stimulator PHA indicates that dietary lactoferrin may specifically affect cells related to an LPS-stimulated response such as B cells or other Toll-like receptor 4 (TLR4) receptor positive cells.

Lactoferrin delivered ex vivo did not attenuate IL-6 production in response to LPS stimulation. FIG. 2B. This indicates that lactoferrin supplementation would not inhibit an innate response to LPS-carrying bacteria. This also indicates that the lactoferrin did not directly interact with LPS such that the presence of LPS in the feeding environment likely does not affect the ability of cells from an individual fed lactoferrin to experience an enhanced cytokine response.

Dietary lactoferrin increased IL-6 production, but was unaffected by ex vivo lactoferrin stimulation. FIG. 2C. This indicates that lactoferrin must be orally administered to be effective and may not be effective if administered via another route such as intravenous or subcutaneously or in any other manner.

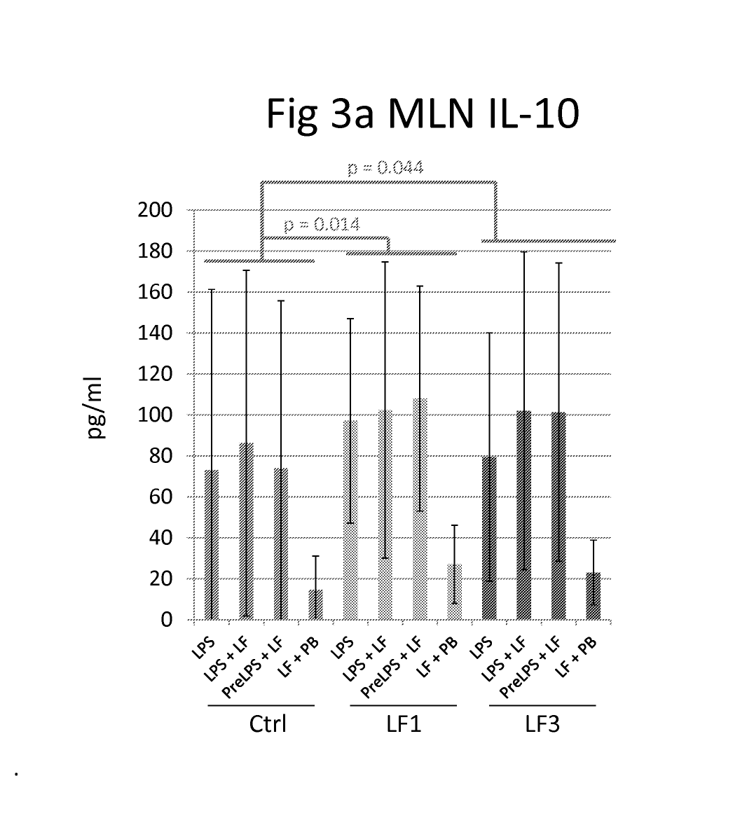

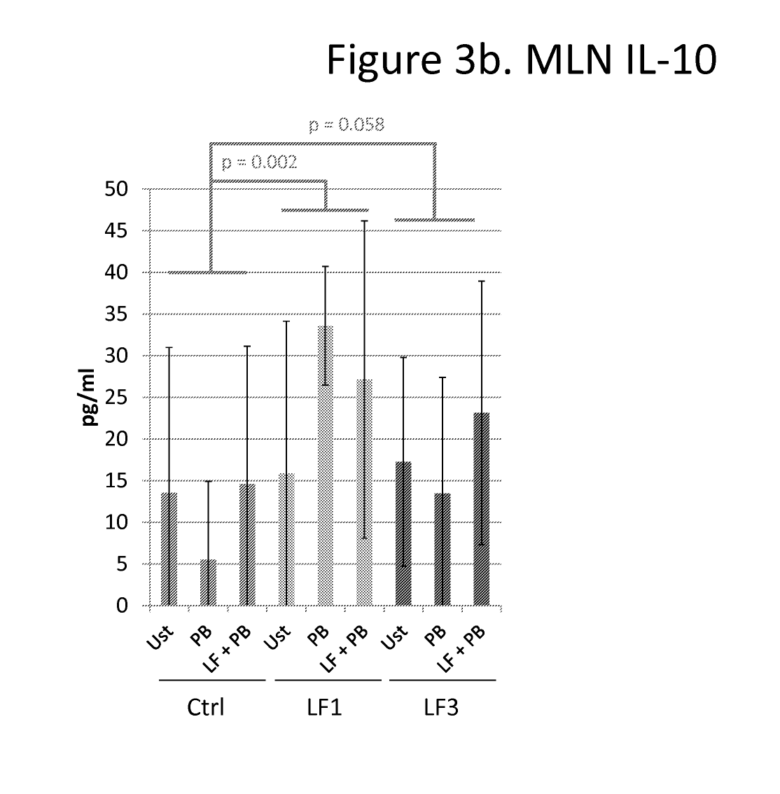

In mesenteric lymph node cells, dietary lactoferrin increased IL-10 production by ex vivo LPS-stimulated cells. Lactoferrin delivered ex vivo did not attenuate IL-10 production in response to LPS stimulation. FIGS. 3A and 3B. This again demonstrates that the lactoferrin must be orally fed to be effective and that lactoferrin did not directly interact with LPS meaning that the presence of LPS in the feeding environment likely does not affect the ability of cells from an individual fed lactoferrin to experience an enhanced cytokine response.

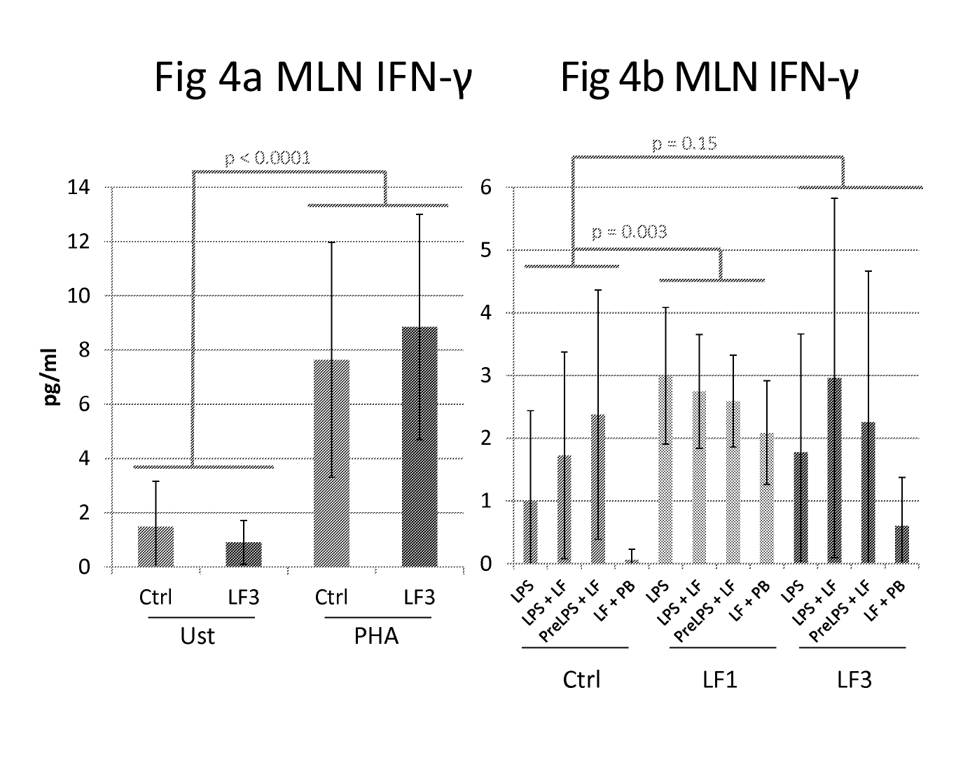

In mesenteric lymph node cells, dietary lactoferrin increased IFN-γ by ex vivo LPS-stimulated cells, but not PHA-stimulated cells. FIG. 4A. Lactoferrin delivered ex vivo did not attenuate IFN-γ production in response to LPS stimulation. FIG. 4B.

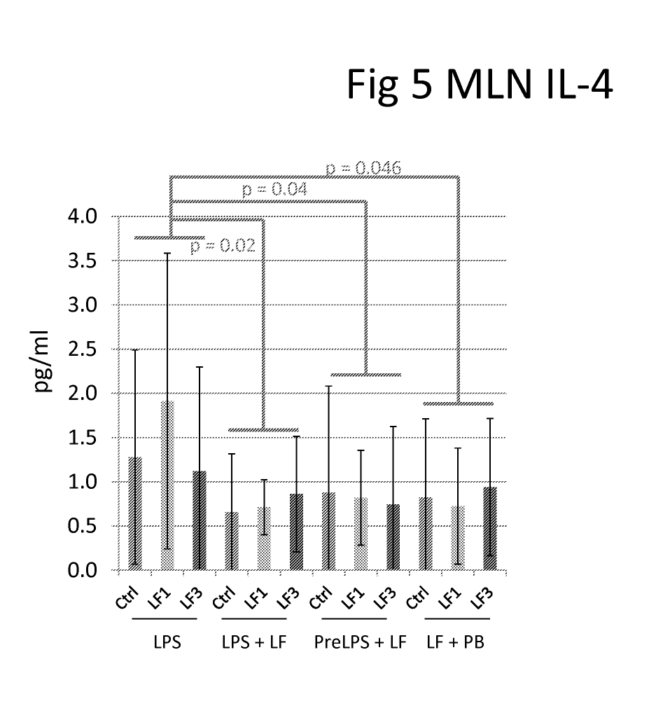

In mesenteric lymph node cells, LPS-stimulated production of IL-4 was attenuated by ex vivo stimulation with lactoferrin. FIG. 5. In contrast to the other effects of lactoferrin on cytokine production, where dietary exposure was necessary to increase cytokine production, the results here indicate that other delivery methods of lactoferrin, such as intravenous, may be more effective at reducing IL-4 production in response to exposure with LPS-positive bacteria than dietary lactoferrin exposure.

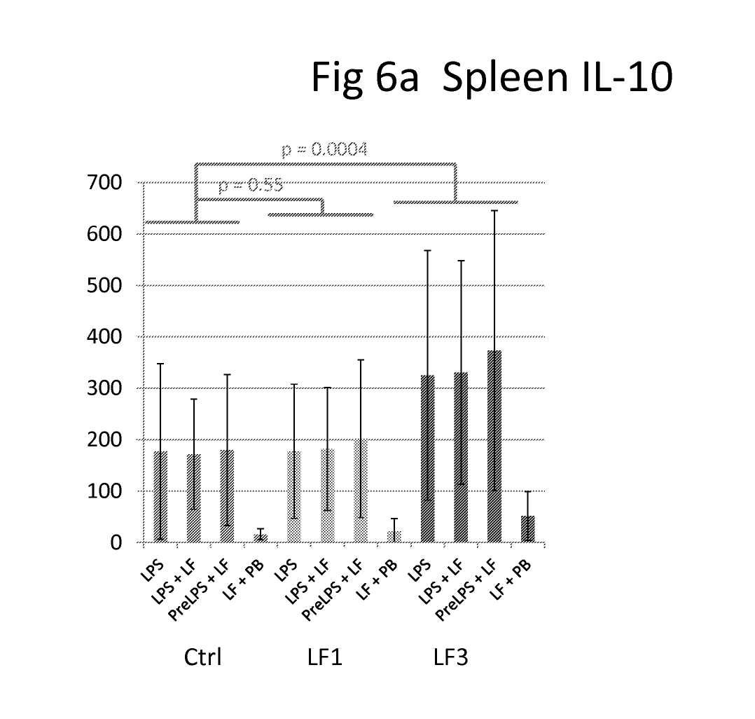

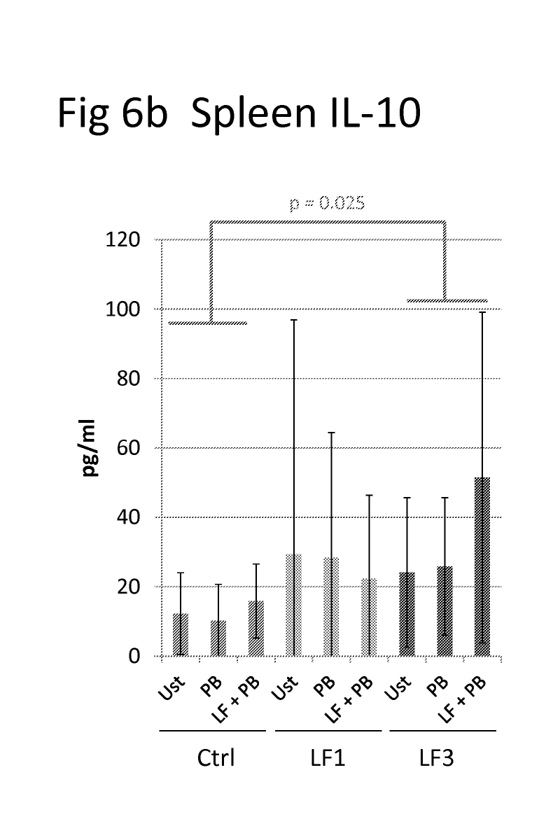

In spleen cells dietary lactoferrin increased IL-10 production by ex vivo LPS-stimulated cells. Lactoferrin delivered ex vivo did not attenuate IL-10 production in response to LPS stimulation. FIGS. 6A and 6B.

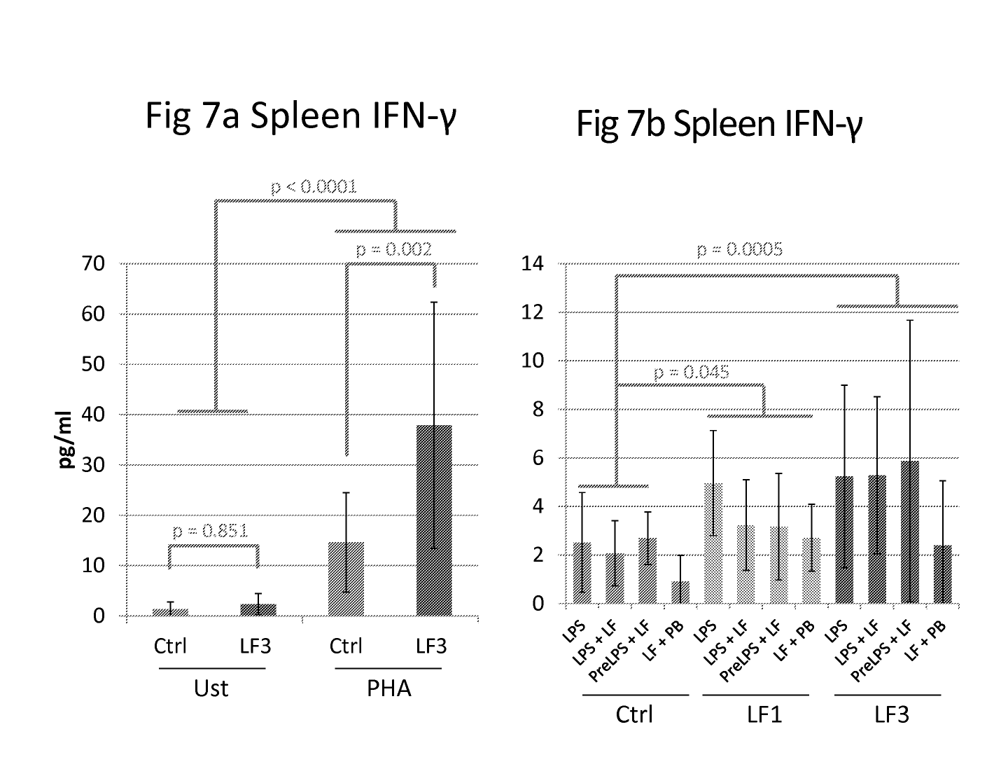

In spleen cells dietary lactoferrin increased IFN-γ by ex vivo LPS-stimulated cells. FIG. 7A. Lactoferrin delivered ex vivo did riot attenuate IFN-γ production in response to LPS stimulation. FIG. 7B.

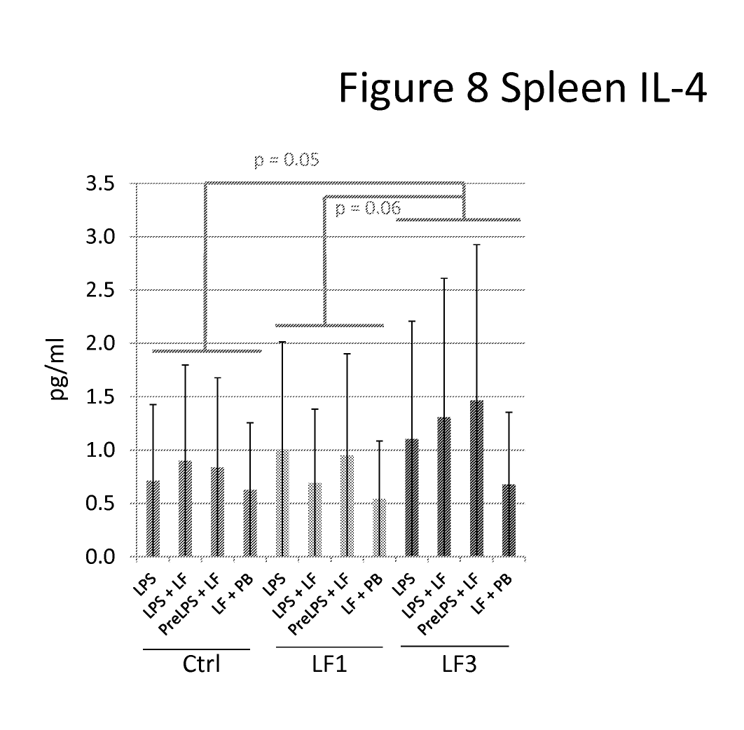

In spleen cells, LPS-stimulated production of IL-4 was increased with dietary lactoferrin. FIG. 8.

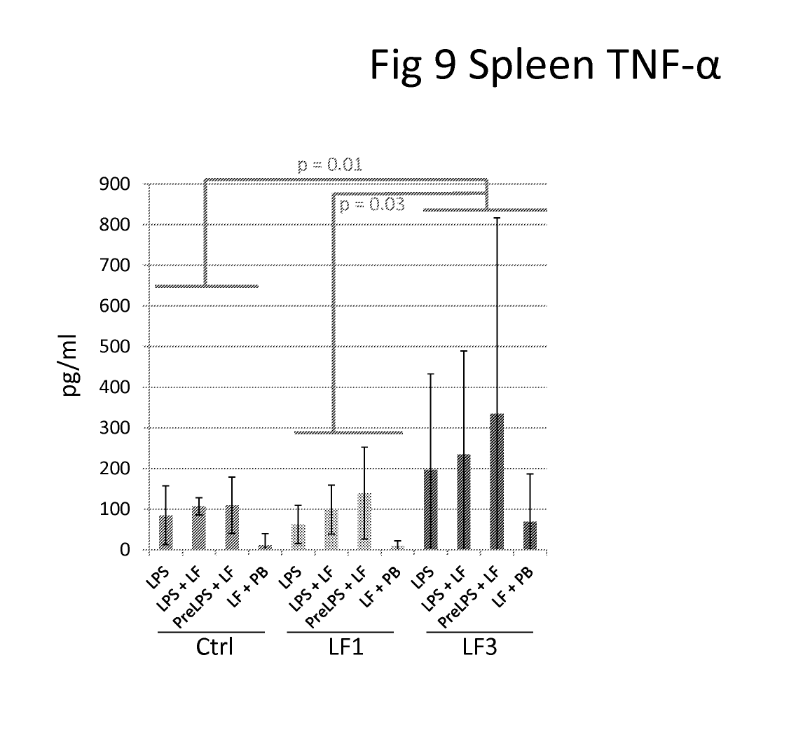

In spleen cells, dietary lactoferrin increased TNF-α by ex vivo LPS-stimulated cells. Lactoferrin delivered ex vivo did not attenuate TNF-α production in response to LPS stimulation. FIG. 9.

In spleen cells IL-12p40 increased with ex vivo PHA stimulation. In contrast to the other effects of dietary lactoferrin where cytokine production was increased in response to LPS-stimulation but not PHA-stimulation, splenic IL-12p40 production was increased with PHA stimulation indicating that dietary lactoferrin also affects T cells.

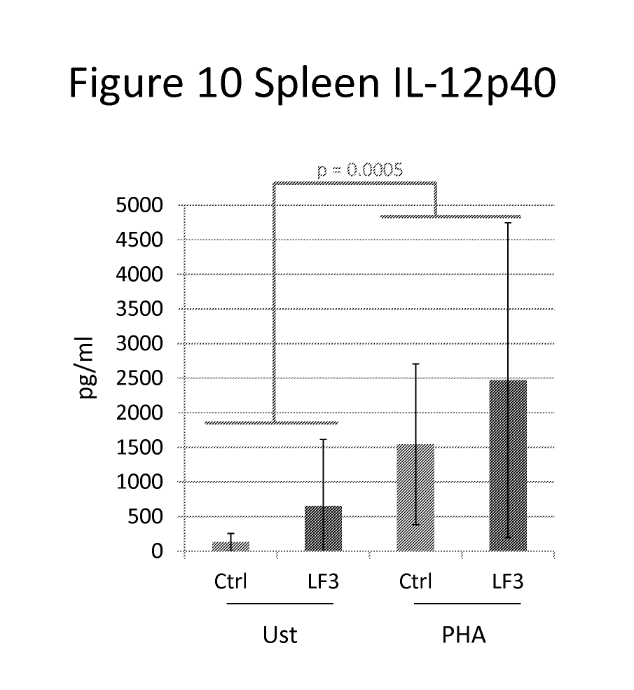

Dietary lactoferrin tended to increase IL-12p40 production. FIG. 10.

Table 1 summarizes the range of cytokines detected in the experiments above.

TABLE 1
Cytokine range (pg/ml)

| Cytokine | Tissue | Unstim | LPS | PHA |
|---|---|---|---|---|
| IFN-gamma | MLN | 0-6 | 0-8 | 4-13 |
| | Spleen | 0-20 | 0-15 | 5-65 |
| IL-6 | MLN | 0-900 | 0-1300 | 60-1000 |
| | Spleen | 0-800 | 0-1200 | 0-400 |
| IL-10 | MLN | 0-100 | 0-250 | 750-3250 |
| | Spleen | 0-200 | 50-720 | 500-6900 |
| TNF-alpha | MLN | 0-380 | 0-400 | 50-200 |
| | Spleen | 0-250 | 0-1400 | 100-300 |
| IL-12p40 | MLN | 0-500 | 0-830 | 50-1000 |
| | Spleen | 0-2000 | 0-5500 | 300-5900 |
| IL-4 | MLN | 0-2.5 | 0-4 | 40-220 |
| | Spleen | 0-3 | 0-3.5 | 70-119 |

Overall the immune response was increased under unstimulated and stimulated conditions mainly when lactoferrin was administered in the diet (oral administration), hut not when lactoferrin was added to directly to cells in culture. This suggests that the method of delivery, via the diet, is necessary for the lactoferrin to be an effective immunostimulant under the condition where the individual is newborn, colostrum-deprived, immunocomprimised or in any of the conditions stated previously. The lack of an effect of ex vivo stimulation with lactoferrin when co-stimulated with ex vivo LPS indicates that environmental LPS will not interfere with the use or applicability of the methods and compositions of the invention. Dietary lactoferrin increased baseline production of many cytokines as well as improving the cytokine response to LPS stimulation indicating a likely improved response to bacterial stimulation in individuals given dietary lactoferrin at the described concentrations, increased levels of circulating immunoglobulins improve protection to both bacteria and viruses. Taken together these data indicate that dietary administration of lactoferrin will result in an improved immune response specifically in immunocompromised, colostrum-deprived individuals or individuals with similar immune insufficiency.

We claim:

1. A method of increasing immune cell function in a newborn mammal that has not consumed any colostrum or breast milk comprising administering an infant formula comprising about 1.0 to about 10 g/L of bovine lactoferrin to the newborn mammal.

2. The method of claim 1, wherein the infant formula comprises about 1.0 to about 3.6 g/L of or about 3.6 to about 5.0 g/L of bovine lactoferrin.

3. The method of claim 1, wherein the newborn mammal has immature immune function, is permanently immunocompromised, or is temporarily immunocompromised.

4. The method of claim 1, wherein the newborn is fully gestated.

5. The method of claim 1, wherein the newborn mammal is born prematurely.

6. The method of claim 1, where in the newborn mammal is just born to 1 hour old.

7. The method of claim 1, wherein the newborn mammal is just born to about 5 hours old or just born to about 1 year old.

8. The method of claim 1, wherein the newborn mammal has a primary or secondary immunodeficiency.

9. The method of claim 8, wherein the primary or secondary immunodeficiency is a B-cell defect, a T-cell disorder, a combined B-cell and T-cell defect, a natural killer cell defect, a phagocytic cell defect, a complement system deficiency, malnutrition, use of immunosuppressive medications, cancer, a chronic infection, diabetes, a hepatic insufficiency, hepatitis, lymphangiectasia, aplastic anemia, graft v. host disease, sickle cell disease, radiation therapy, splenectomy, cytomegalovirus, Epstein-Barr virus, measles virus, varicella-zoster virus, nephrotic syndrome, renal insufficiency, uremia, or AIDS.

10. The method of claim 1, wherein the newborn mammal is a human having an IgG concentration of less than about 7 g/L.

11. The method of claim 1, wherein the mother of the newborn infant has placental abnormalities, hypergammaglobulinemia, HIV infection, placental malaria, a humoral immunedeficiency, or other infection or disease that causes reduced placental transfer of IgG.

12. The method of claim 1, wherein the increase in immune cell function is an increase in total serum immunoglobulin concentration or an increase in cytokine secretion from immune cells or a combination thereof.

13. The method of claim 12, wherein the increase in cytokine secretion is an increase in IL-6, IL-10, IFN-gamma, IL-4, TNF-alpha, IL12p40, or a combination thereof.

14. The method of claim 1, wherein the newborn mammal is a human, pig, canine, equine, or feline.

15. A method of increasing immune cell function in a newborn mammal that has consumed colostrum or breast milk for a period not exceeding 1 week after birth, comprising administering an infant formula comprising about 1.0 to about 10 g/L of bovine lactoferrin to the newborn mammal.

16. A method of increasing immune cell function in a newborn mammal that has consumed a total not exceeding about 2 ml of colostrum, or a total not exceeding about 10 ml of breast milk, or a total not exceeding about 2 ml of colostrum and a total not exceeding about 10 ml of breast milk comprising administering an infant formula comprising about 1.0 to about 10 g/L of bovine lactoferrin to the newborn mammal.
